(19) United States
(12) Patent Application Publication
(10) Pub. No.: US 2016/0303307 A1
(43) Pub. Date: Oct. 20, 2016

(54) ULTRASOUND-GUIDED VASCULAR DEVICE FOR EXTRACORPOREAL MEMBRANE OXYGENATION

(71) Applicant: THE CHARLOTTE-MECKLENBURG HOSPITAL AUTHORITY D/B/A CAROLINAS HEALTHCARE SYSTEM, Charlotte, NC (US)

(72) Inventors: Jeko Metodiev Madjarov, Charlotte, NC (US); Allen C. Heffner, Charlotte, NC (US); Liam P. Ryan, Charlotte, NC (US)

(21) Appl. No.: 15/103,142

(22) PCT Filed: Dec. 9, 2014
(86) PCT No.: PCT/US2014/069308

§ 371 Date: Jun. 9, 2016

Related U.S. Application Data
(60) Provisional application No. 61/914,092, filed on Dec. 10, 2013.

(51) Int. Cl. A61M 1/36; A61B 8/08; A61B 8/00; A61B 8/12

(52) U.S. Cl. CPC A61M 1/3666; A61B 8/12; A61B 8/0891; A61B 8/4472; A61B 8/445; A61B 8/0883; A61B 8/0841; A61B 8/4477; A61M 2205/3375

(57) ABSTRACT

A vascular device and method for positioning a vascular device within a patient's vasculature for use in extracorporeal membrane oxygenation (ECMO) are described that make use of one or more ultrasound transducers supported by the vascular device to visualize the location and orientation of the vascular device. An ultrasound transducer may be provided at a port of the vascular device that infuses oxygenated blood into the patient's vasculature to allow the operator of the vascular device to align the port with a particular structure in the patient's body, such as the tricuspid valve of the heart. Another ultrasound transducer may additionally or alternatively be provided at a distal end of the vascular device to facilitate insertion of the vascular device with minimal injury or trauma to the patient and to allow advancement of the device to the correct location. The imaging may be continuous and may be provided in real-time.

### FIELD OF THE INVENTION

The present invention relates generally to methods and vascular devices for use in extracorporeal membrane oxygenation.

### BACKGROUND

Extracorporeal membrane oxygenation (ECMO) is a technique of providing both cardiac and respiratory support oxygen to patients who are suffering from conditions that prevent their heart and lungs from functioning properly, for example, as a result of disease or trauma. In cases where the patient is expected to recover and resume proper heart and lung function, ECMO may be used as a temporary measure to allow the patient's body to heal. For example, ECMO may be used in the case of cardiac arrest or refractory cardiogenic shock, as a bridge to cardiac transplantation or placement of a ventricular assist device, and in other situations in which there is a certain likelihood that the patient will recover and is expected to have a reasonable quality of life.

To prepare a patient for ECMO, cannulae are inserted in the patient's vasculature and connected to an ECMO circuit to extract deoxygenated blood from the patient, oxygenate the blood, and then reintroduce the now oxygenated blood into the patient's body. In veno-arterial (VA) ECMO, a venous cannula is usually placed in the right common femoral vein for extraction of the deoxygenated blood, and an arterial cannula is usually placed into the right femoral artery for infusion of the oxygenated blood into the patient's system. The tip of the femoral venous cannula may, for example, be positioned and maintained near the junction of the inferior vena cava and right atrium, while the tip of the femoral arterial cannula is maintained in the iliac artery. In veno-venous (VV) ECMO, venous cannulae may be placed in the right common femoral vein for extraction and in the right internal jugular vein for infusion.

### BRIEF SUMMARY

As noted above, one or more cannulae are typically used for extraction and infusion during ECMO. Accordingly, to prepare a patient to undergo ECMO, the cannula(e) are generally inserted into the patient's vasculature and positioned percutaneously, such as by using the Seldinger technique to advance the cannula(e) to an appropriate position. Correct positioning of the cannula or cannulae is important for obtaining optimal oxygen delivery and achieving the best results for the patient.

Accordingly, devices and methods are provided in accordance with example embodiments for vascular procedures, such as for facilitating an ECMO procedure. In some embodiments, a vascular device is provided that is configured for placement within a patient's vasculature. The vascular device may comprise a tubular body defining a first lumen, a first lumen port, a second lumen, and a second lumen port. The first lumen port may be in fluid communication with the first lumen, and the first lumen may be configured to extract deoxygenated blood from the patient's vasculature via the first lumen port and convey the deoxygenated blood to a machine for oxygenation. Similarly, the second lumen port may be in fluid communication with the second lumen, and the second lumen may be configured to convey oxygenated blood from the machine and infuse the oxygenated blood into the patient's vasculature via the second lumen port. The vascular device may further comprise an ultrasound transducer supported by the tubular body and disposed proximate the second lumen port.

In some cases, the tubular body may define a plurality of first lumen ports. For example, the tubular body may define 2 to 4 first lumen ports on a distal side of the second lumen port and/or 2 to 6 first lumen ports on a proximal side of the second lumen port. The vascular device may further be configured to be positioned proximate a right atrium of the patient's body such that oxygenated blood flowing from the second lumen port is directed at the tricuspid valve of the heart.

Moreover, in some embodiments, the tubular body may define a distal end having an opening in fluid communication with the first lumen, such that deoxygenated blood is received into the first lumen from the patient's vasculature via the opening of the distal end and is conveyed to the machine for oxygenation.

In some cases, the ultrasound transducer may be a second lumen port ultrasound transducer, and the vascular device may further comprise a distal end ultrasound transducer supported by the tubular body and disposed proximate a distal end of the tubular body. The distal end ultrasound transducer may be configured to transmit and receive ultrasound signals so as to provide an image of an interior of the patient's blood vessel within which the vascular device is disposed proximate the location of the distal end, such that a longitudinal position of the vascular device may be determined with respect to the patient's blood vessel and surrounding anatomical structures and such that the longitudinal position of the vascular device may be adjusted by an operator of the vascular device based on the image provided by at least one of the distal end ultrasound transducer or the second lumen port ultrasound transducer to achieve optimal perfusion of the patient's body.

Additionally or alternatively, the vascular device may be configured to be positioned such that a distal end of the tubular body is disposed proximate a superior vena cava of the patient's heart and a proximal portion of the vascular device is disposed proximate an inferior vena cava of the patient's heart.

The ultrasound transducer may be wireless. In some cases, the ultrasound transducer may be integrally formed with the tubular body.

In still other embodiments, a method for positioning a vascular device within a patient's vasculature is provided. The method may include advancing a vascular device from an entry point through the patient's vasculature towards the patient's heart. In this regard, the vascular device may comprise a tubular body defining a first lumen; a first lumen port in fluid communication with the first lumen, wherein the first lumen is configured to extract deoxygenated blood from the patient's vasculature via the first lumen port and convey the deoxygenated blood to a machine for oxygenation; a second lumen; and a second lumen port in fluid communication with the second lumen, wherein the second lumen is configured to convey oxygenated blood from the machine and introduce the oxygenated blood into the patient's vasculature via the second lumen port. The vascular device may further comprise an ultrasound transducer supported by the tubular body and disposed proximate the second lumen port.

According to embodiments of the method, the vascular device may be positioned proximate the patient's right atrium, and an image may be received of an interior of the patient's blood vessel within which the vascular device is disposed proximate the location of the second lumen port of the vascular device from the ultrasound transducer supported by the vascular device. The vascular device may then be rotated based on the image received, such that the second lumen port is substantially aligned with the tricuspid valve of the patient's heart to achieve optimal perfusion of the patient's body.

In some cases, the tubular body may define a plurality of first lumen ports. The tubular body may define 2 to 4 first lumen ports on a distal side of the second lumen port, and/or the tubular body may define 2 to 6 first lumen ports on a proximal side of the second lumen port. Additionally or alternatively, the tubular body may define a distal end having an opening in fluid communication with the first lumen, such that deoxygenated blood is received into the first lumen from the patient's vasculature via the opening of the distal end and is conveyed to the machine for oxygenation.

In some embodiments, the ultrasound transducer may be a second lumen port ultrasound transducer, and the vascular device may further comprise a distal end ultrasound transducer supported by the tubular body and disposed proximate a distal end of the vascular device. The method may further comprise receiving a second image of an interior of the patient's blood vessel within which the vascular device is disposed proximate the distal end of the vascular device from the distal end ultrasound transducer. In some cases, a longitudinal position of the vascular device may be adjusted based on the images provided by the distal end ultrasound transducer and the second lumen port ultrasound transducer to achieve optimal perfusion of the patient's body.

Moreover, the vascular device may be positioned such that a distal end of the vascular device is disposed proximate a superior vena cava of the patient's heart and a proximal portion of the vascular device is disposed proximate an inferior vena cava of the patient's heart.

The ultrasound transducer may be wireless in some cases. Additionally or alternatively, the ultrasound transducer may be integrally formed with the tubular body.

In still other embodiments, a vascular device is provided that is configured for placement within a patient's vasculature. The vascular device may comprise a tubular body defining a lumen and a port in fluid communication with the lumen. The lumen may be configured to extract deoxygenated blood from the patient's vasculature or infuse oxygenated blood into the patient's vasculature and may be in fluid communication with a machine for oxygenation. The vascular device may further comprise an ultrasound transducer supported by the tubular body and disposed proximate the port.

In still other embodiments, a vascular device is provided that is configured for placement within a patient's vasculature, where the device includes a tubular body having a distal end and defining a lumen and a port in fluid communication with the lumen. The lumen may be configured to extract deoxygenated blood from the patient's vasculature or infuse oxygenated blood into the patient's vasculature and may be in fluid communication with a machine for oxygenation. The vascular device may further include an ultrasound transducer supported by the tubular body and disposed proximate the distal end.

### BRIEF DESCRIPTION OF THE DRAWINGS

Having thus described the invention in general terms, reference will now be made to the accompanying drawings, which are not necessarily drawn to scale, and wherein:

FIG. 1 shows a schematic representation of a conventional ECMO circuit;

FIG. 2 shows a schematic representation of an ECMO circuit in accordance with an exemplary embodiment of the present invention;

FIG. 3 shows a simplified sectional view of a heart with a vascular device in place for facilitating ECMO in accordance with an exemplary embodiment of the present invention;

FIG. 4 shows a vascular device having a first lumen and a second lumen with an ultrasound transducer proximate the second lumen port in accordance with an exemplary embodiment of the present invention;

FIG. 4A shows a cross-sectional representation of a proximal portion of the vascular device of FIG. 4 in accordance with an exemplary embodiment of the present invention;

FIG. 4B shows a cross-sectional representation of a distal portion of the vascular device of FIG. 4 in accordance with an exemplary embodiment of the present invention;

FIG. 5 illustrates a close-up view of the second lumen port of FIG. 4 with a portion of the tubular body wall removed in accordance with another exemplary embodiment of the present invention;

FIG. 6 shows a vascular device having a first lumen and a second lumen with an ultrasound transducer proximate the second lumen port and an ultrasound transducer proximate a distal end of the vascular device in accordance with an exemplary embodiment of the present invention;

FIG. 7 illustrates a close-up view of the distal end of the vascular device of FIG. 6 with a portion of the tubular body wall removed in accordance with another exemplary embodiment of the present invention; and

FIG. 8 shows a simplified sectional view of a heart with the vascular device of FIG. 6 in place for facilitating ECMO in accordance with an exemplary embodiment of the present invention.

### DETAILED DESCRIPTION

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

As used herein, the terms “distal” and “distally” refer to a location farthest from a reference point, such as an entry point into a patient's vasculature and/or an operator of the vascular device. Similarly, the terms “proximal” and “proximally” refer to a location closest to the reference point. Furthermore, although the examples described herein refer to a vascular device for use during ECMO, embodiments of the described invention may be used in various other situations and for other procedures requiring a vascular device to be advanced through a patient's vasculature to a target site in the patient's body.

Veno-Venous Extracorporeal Membrane Oxygenation (VV-ECMO) support is quickly becoming the standard of care for pulmonary support in patients with severely limited gas exchange and/or diminished ventilator capacity who fail to respond to optimal medical management (e.g., bi-level ventilation, nitric oxide, etc.). As a consequence of the medical field's continually improving ability to stabilize patients with severe multisystem organ dysfunction, this clinical scenario is becoming increasingly common and most often develops in the context of acute respiratory distress syndrome (ARDS), transfusion related acute lung injury (TRALI), aspiration pneumonitis, and pulmonary contusion.

Survival rates for severe manifestations of the aforementioned syndromes have historically remained well below 20%. Refinement of VV-ECMO techniques optimized for these acute pulmonary syndromes has improved survival rates to 80% or better at experienced centers. There are, however, very few centers in the United States equipped to offer VV-ECMO to their patient population. This is particularly true in rural and underserved areas. In these cases, the only realistic chance of survival lies in expeditious transfer to a center equipped for prolonged ECMO support. Unfortunately, many patients suffering from these pulmonary syndromes are not stable enough for transfer and ultimately succumb to a survivable disease process.

At many hospitals that do not offer ECMO support, the limiting factor in providing this service is the absence of surgeons and cardiologists familiar with cannula insertion techniques, all of which require intra-procedural access to transthoracic and/or transesophogeal echocardiography (ideally 3D-echocardiography) and fluoroscopy/angiography. Even centers with easy access to skilled operators and image-guidance are often limited in their ability to provide ECMO support, as the patients are frequently too unstable to travel to the cardiac catheterization lab or hybrid operating suite. In these cases, blind cannula insertion or insertion with minimal image guidance is the only realistic option and carries an extremely high risk of cannula malpositioning and vascular perforation.

Moreover, even when conventional imaging techniques are used to achieve the initial placement of the cannulae, the flow of blood through the vasculature and the pumping action of the heart often cause the cannulae to change position. Without constant or repeated imaging, which is impractical, if not impossible, using conventional imaging techniques, the efficiency of ECMO can be negatively affected. The displacement of the cannulae can go undetected for a length of time (e.g., until the effects are manifested in the patient's condition) and usually results in further deterioration of the patient's condition.

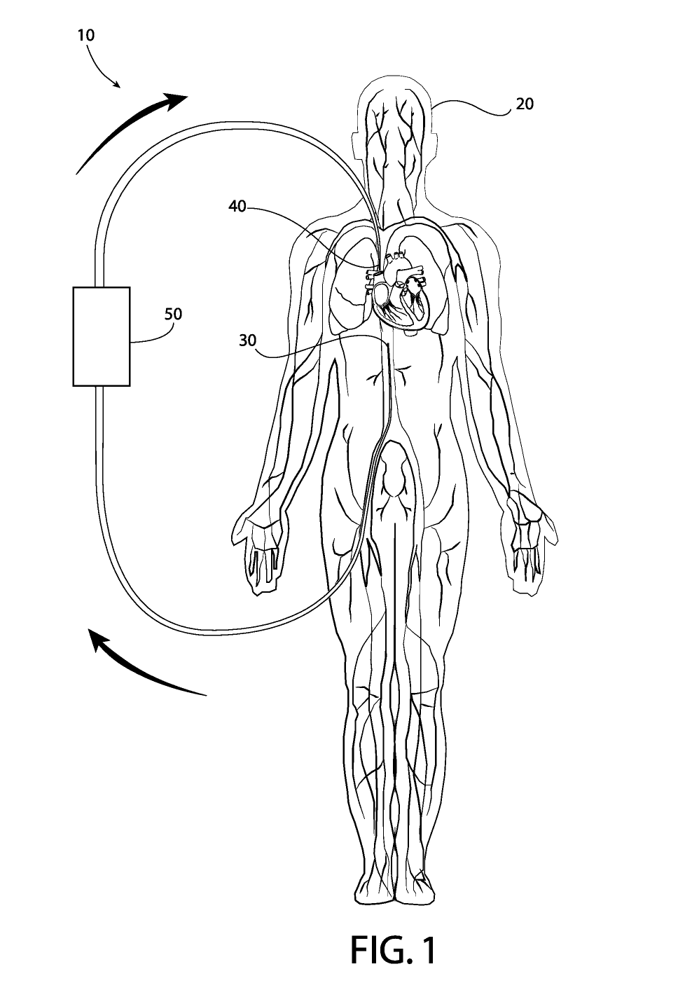

FIG. 1 depicts an example of a conventional ECMO circuit 10 that may be used to oxygenate a patient's blood, such as may be used for VV ECMO. In a conventional ECMO procedure, blood may be extracted from the patient's body using a venous cannula 30 (shown schematically in FIG. 1) positioned in the patient's vasculature, such as in the right common femoral vein via an entry point in the groin. Another venous cannula 40 may be inserted via an entry point in the patient's neck and positioned in the patient's right internal jugular vein for infusion. Thus, deoxygenated blood may flow into the cannula 30 and be passed through an ECMO machine 50, which does the work of the patient's heart and lungs. Oxygenated blood may then be pumped from the ECMO machine 50 back into the patient's body via the cannula 40. Although one particular example of an ECMO circuit is shown in FIG. 1, other ECMO circuits may be used in other situations, such as depending on the condition of the patient's body, the patient's age, the disease or cause of the impairment, the expected duration of ECMO, etc.

Regardless of the specific configuration of the ECMO circuit, the advancement and positioning of the cannulae can be one of the most difficult aspects of the procedure. The condition of the patient's veins and arteries may vary, and in some cases portions of the patient's vessel walls may be weak or brittle due to disease or age. A misguided cannula may injure or tear the vessel wall or nearby structures, which may cause internal bleeding and make the patient's already serious condition even graver, and potentially fatal. Moreover, improper positioning of the cannulae, such as by not advancing the cannulae far enough or having the wrong orientation can significantly impact the efficiency of ECMO. For example, infusing the oxygenated blood at the wrong location or directing the flow of oxygenated blood in the wrong direction may create backflow and/or unnecessarily mingle oxygenated blood with deoxygenated blood. As a result, the patient's organs may not receive blood with a high enough oxygen content, which may cause necrosis or severely limit the patient's ability to heal.

Accordingly, embodiments of a vascular device are described which allow for real-time ultrasound imaging of one or more locations near the vascular device from within the patient's blood vessel to allow the operator of the vascular device to visualize the precise location and orientation of the vascular device without the need for additional specialized practitioners or equipment. In this regard, embodiments of the vascular device include one or more ultrasound transducers carried by the vascular device, such that the real-time surroundings of the vascular device can be monitored and the position of the vascular device can be adjusted as needed to obtain optimal ECMO results. Embodiments of the vascular device may thus allow for initiation of ECMO support in community centers that lack the technical and imaging support required for current techniques and technology. Furthermore, embodiments of the vascular device may allow for bedside, image-guided ECMO cannulation without radiation or contrast and may dramatically improve the safety of cannula insertion, even at highly experienced centers.

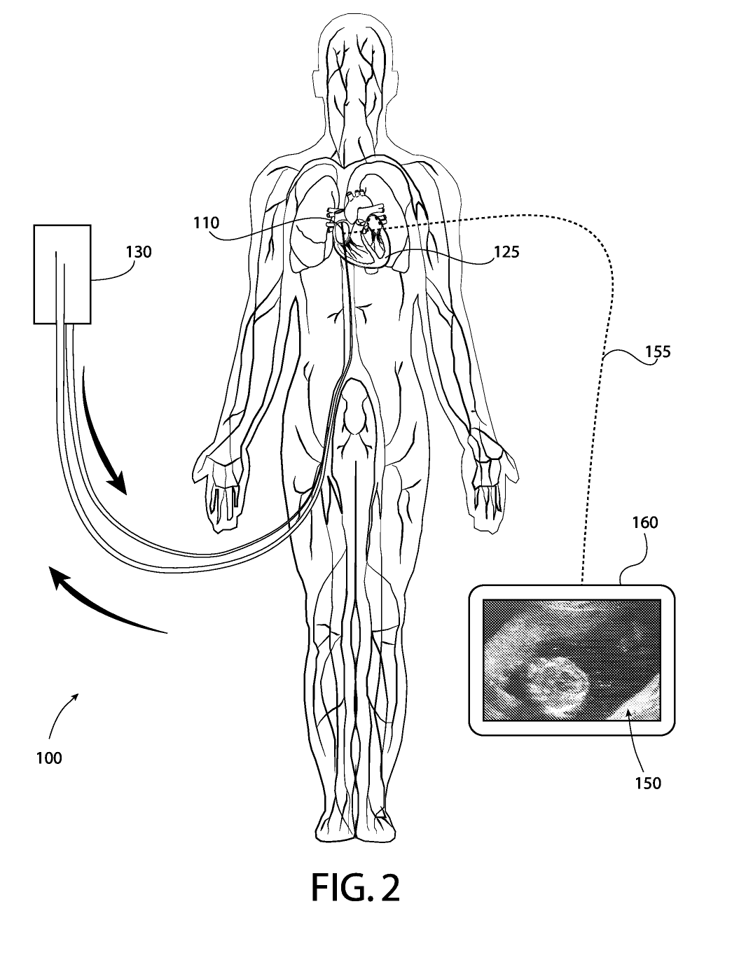

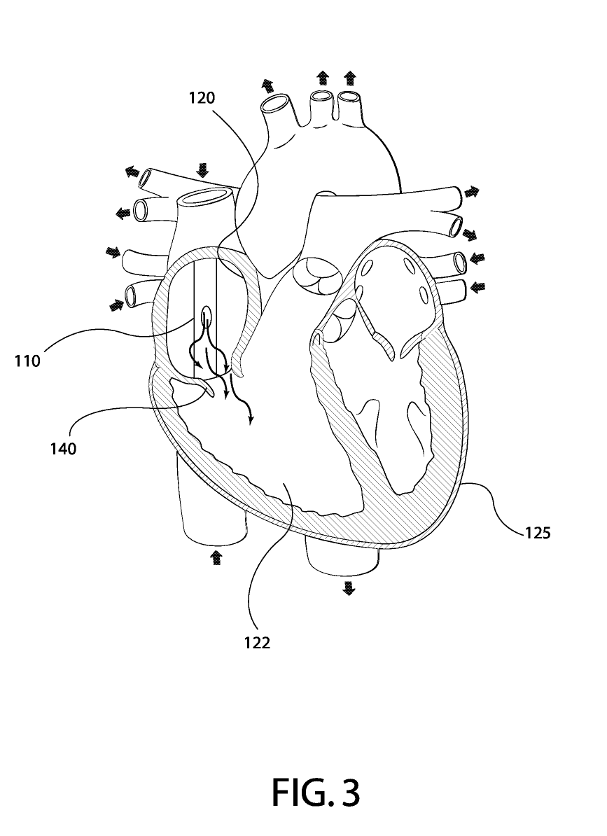

With reference now to FIG. 2, an ECMO circuit 100 is depicted according to an example embodiment of the invention. In the depicted embodiment, and as described in greater detail below, deoxygenated blood is extracted from the patient via a vascular device 110, shown in FIG. 3, placed in the vicinity of the right atrium 120 of the patient's heart 125. The deoxygenated blood may flow out of the patient's body via the vascular device 110 and into an ECMO machine 130, which may perform the work of the patient's heart and/or lungs. Oxygenated blood may then be pumped from the ECMO machine 130 back into the patient's body using a different lumen of the same vascular device 110. As shown in FIG. 3, the vascular device 110 may be positioned within the right atrium 120 such that the oxygenated blood may be directed toward the tricuspid valve 140 of the heart, which separates the right atrium from the right ventricle 122. By accurately positioning the vascular device 110 such that the flow of oxygenated blood is directed toward the tricuspid valve 140, the efficiency of ECMO procedure can be optimized, as a maximum amount of oxygenated blood enters the right ventricle 122 and is advanced through the lungs, other heart chambers, and throughout the rest of the patient's body.

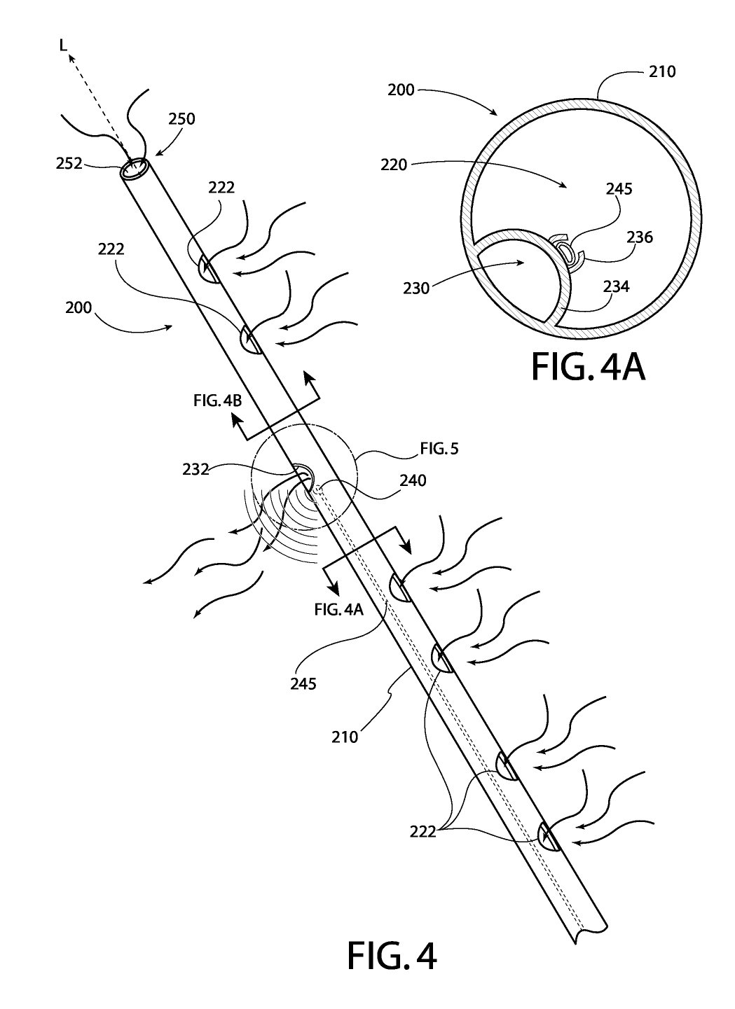

In this regard, the vascular device 110 includes at least one ultrasound transducer (see, e.g., FIG. 4, reference character 240) that is configured to visualize the location of the vascular device, such that the position and/or orientation of the vascular device can be easily determined by an operator of the device and monitored for the duration of the ECMO procedure. As shown in FIG. 2, the ultrasound transducer of the vascular device 110 may transmit an image 150 depicting the inside of the blood vessel within which the vascular device is positioned to a display 160 that is observed by the operator of the vascular device, as represented in FIG. 2 via a dashed line arrow 155. By referring to the image 150 presented on the display 160, the operator can determine, in real-time, the position of the vascular device and make any adjustments necessary to properly position the vascular device and achieve optimal ECMO results.

With reference to FIG. 4, in some example embodiments, a vascular device 200 is shown that is configured for placement within a patient's vasculature for use in ECMO. The vascular device 200 may include a tubular body 210 that defines a first lumen 220 (shown in FIGS. 4A and 4B) and a first lumen port 222 in fluid communication with the first lumen. The first lumen 220 may be configured to extract deoxygenated blood from the patient's vasculature via the first lumen port 222 and to convey the deoxygenated blood to an ECMO machine 130 (shown in FIG. 2) for oxygenation. The tubular body 210 may further define a second lumen 230 and a second lumen port 232 in fluid communication with the second lumen. The second lumen 230 may be configured to convey oxygenated blood from the ECMO machine 130 of FIG. 2 and infuse the oxygenated blood into the patient's vasculature via the second lumen port 232.

The vascular device 200 may further comprise an ultrasound transducer 240 supported by the tubular body. In the illustrated embodiment, the transducer is shown as being disposed proximate the second lumen port 232. The ultrasound transducer 240 may be configured to transmit and receive ultrasound signals so as to provide an image of an interior of the patient's blood vessel within which the vascular device 200 is disposed proximate the location of the second lumen port 232. For example, the ultrasound transducer 240 may be configured to provide a radial intravascular imaging plane. In this way, at least a rotational position of the vascular device 200 may be determined with respect to the patient's blood vessel and surrounding anatomical structures, such that the rotational position of the vascular device may be adjusted by an operator of the vascular device based on the image provided by the ultrasound transducer 240 to achieve optimal perfusion of the patient's body. For example, the operator may rotate the vascular device 200 until the tricuspid valve 140 is seen in the image provided by the ultrasound transducer 240.

In this regard, the term “rotational position” refers to the position of the vascular device 200 as it is rotated about a longitudinal axis L of the tubular body 210 with respect to the blood vessel within which the vascular device 200 is disposed. The term “longitudinal position,” as used herein, refers to the position of the vascular device 200 along the longitudinal axis L of the tubular body 210 as it is advanced or withdrawn with respect to the blood vessel within which the vascular device 200 is disposed.

As shown in FIG. 4, the first lumen 220 may, in some embodiments, extend proximally from a distal end 250 of the tubular body 210. The second lumen 230, in contrast, may extend proximally from the second lumen port 232, where the second lumen port 232 is located proximally from the distal end 250. Thus, the second lumen 230 may be shorter than the first lumen 220 in some embodiments.

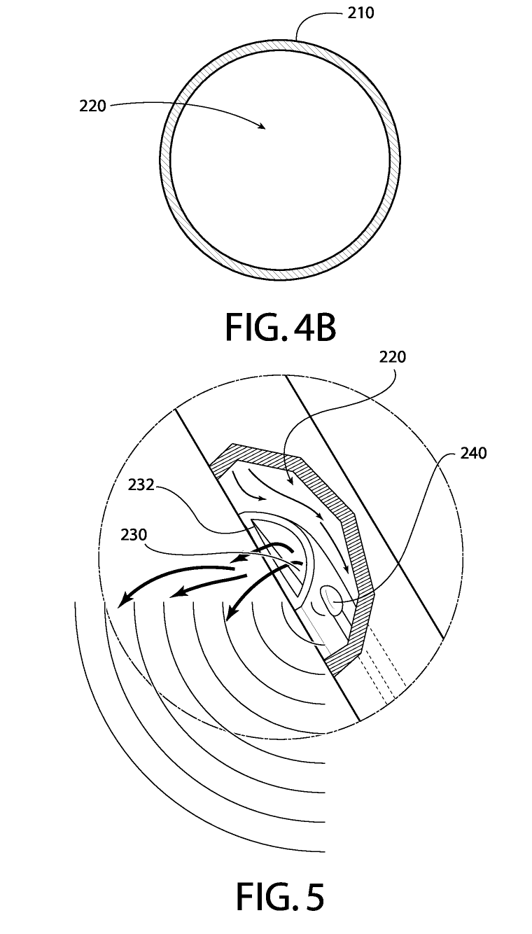

In addition, in some cases, a cross-sectional area of the first lumen 220 may be larger than a cross-sectional area of the second lumen 230 and may vary in cross-sectional area along a length of the device. For example, as shown in FIG. 4A, at a location that is proximal with respect to the second lumen port 240, the first lumen 220 may have a cross-section that is defined by one portion of the cross-section of the tubular body 210, while the second lumen 230 may have a cross-section that is defined by another portion of the cross-section of the tubular body. Distally of the second lumen port 232, however, the first lumen 220 may take up the entire cross-sectional area defined by the tubular body 210. In this way, blood extracted from the blood vessel via one or more distal first lumen ports 222 may flow proximally (e.g., toward the ECMO machine) around the second lumen 230, as depicted in FIG. 5.

In some embodiments, as shown in FIG. 4, the distal end 250 of the tubular body 210 may have an opening 252 that is in fluid communication with the first lumen 220, such that deoxygenated blood is extracted into the first lumen from the patient's vasculature via the opening 252 of the distal end 250 and is conveyed to an ECMO machine for oxygenation. In other embodiments, however, the distal end 250 may be capped. The tubular body 210 may define a plurality of first lumen ports 222 in some cases, and blood flow from each of the first lumen ports may combine within the first lumen 220 as the blood is extracted and conveyed to the ECMO machine. In some embodiments, for example, the tubular body 210 may define 2 to 4 first lumen ports 222 on a distal side of the second lumen port 232. Additionally or alternatively, the tubular body 210 may, in some embodiments, define 2 to 6 first lumen ports 222 on a proximal side of the second lumen port 232. In the embodiment depicted in FIG. 4, 2 first lumen ports 222 are provided on a distal side of the second lumen port 232, and 4 first lumen ports are provided on a proximal side of the second lumen port, in addition to the opening 252 of the distal end 250.

With reference to FIGS. 4 and 4A, in some embodiments, the ultrasound transducer 240 may be integrally formed with the tubular body 210. For example, an inner wall 234 of the tubular body 210 (e.g., a wall separating the first and second lumens 220, 230) may define a support structure 236 configured to attach the transducer 240 and/or transducer leads 245 within the second lumen, such that the ultrasound transducer is an integral, permanent part of the vascular device 200. As another example, the ultrasound transducer 240 may be embedded within a wall of the tubular body 210. In other cases, however, the ultrasound transducer 240 may be a stand-alone ultrasound probe that is inserted into one of the lumens 220, 230 (or into a separate, e.g., third lumen not shown) and may be detachable from the vascular device 200, such that the vascular device may be used with or without the transducer.

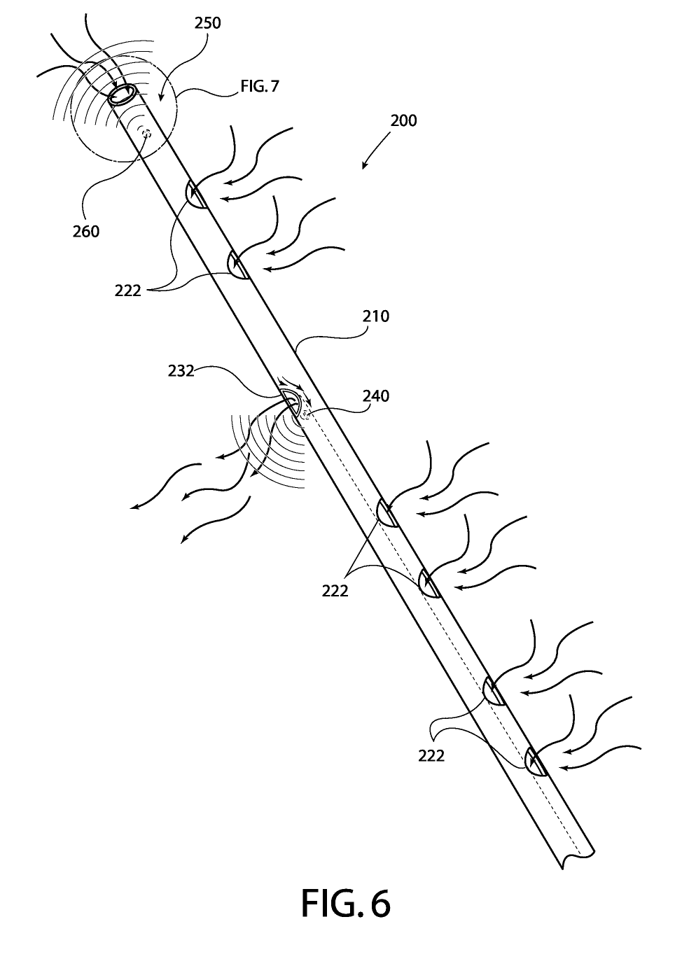

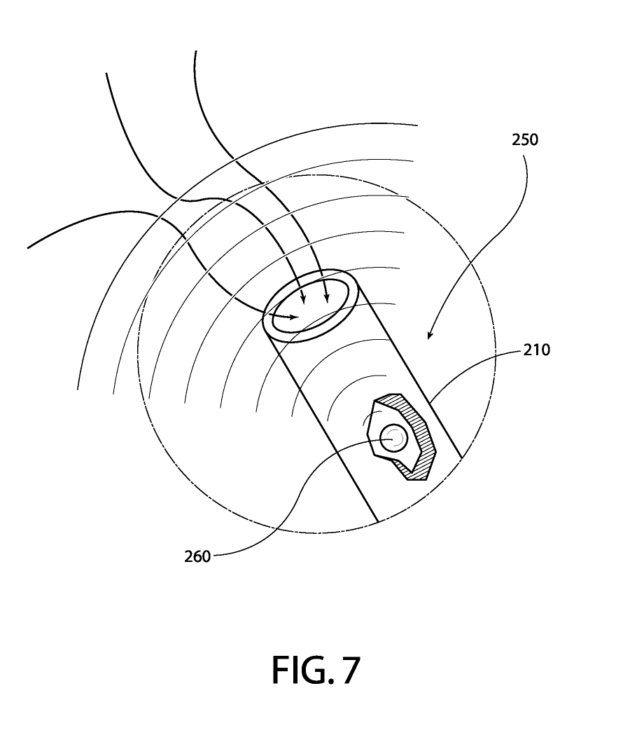

In the embodiment shown in FIG. 4 the ultrasound transducer 240 has wire leads 245 configured to power the transducer and to transmit the ultrasound image information to an external display (such as the display 160 shown in FIG. 2). Alternatively, in some embodiments, such as the embodiment depicted in FIGS. 6 and 7 and described above, the ultrasound transducer 240 may be a wireless ultrasound transducer. In this regard, the wireless ultrasound transducer of FIGS. 6 and 7 may be configured such that the transducer is powered and can transmit image information wirelessly, such as over a wireless network connection.

In some embodiments, at least the tubular body 210 of the vascular device 200 may be made of a biocompatible polymer, including, for example, polyurethane and/or silicone. The vascular device may have various dimensions depending on the size of the patient (e.g., adult or pediatric), the condition of the patient's vasculature, and the specific ECMO procedure to be performed, among other factors. For example, an overall diameter of the tubular body 210 may be between approximately 12 Fr (4 mm) to approximately 33 Fr (11 mm), and the length of the tubular body (e.g., measured from the distal end 250 to the most proximal first lumen port 222) may be between approximately 10 cm to approximately 35 cm long or longer. The total insertion length of the vascular device 200, in some embodiments, may be between approximately 35 cm long to approximately 70 cm long. In particular, in the case of a vascular device 200 configured to be used for an ECMO procedure in which the device is inserted via the femoral vein through an entry point in the groin, the overall diameter of the tubular body 210 may be between approximately 23 Fr (7⅔ mm) to approximately 29 Fr (9⅔ mm), and the total insertion length of the vascular device may be approximately 40 to 45 cm.

With reference now to FIGS. 6 and 7, in some embodiments, multiple ultrasound transducers may be provided so as to allow the visualization of both the rotational position of the vascular device 200 and the longitudinal position of the vascular device to facilitate insertion of the device and alignment of the second lumen port 232 with the tricuspid valve 140. In this regard, the ultrasound transducer 240 may be considered a second lumen port ultrasound transducer 240, and the vascular device 200 may further comprise a distal end ultrasound transducer 260.

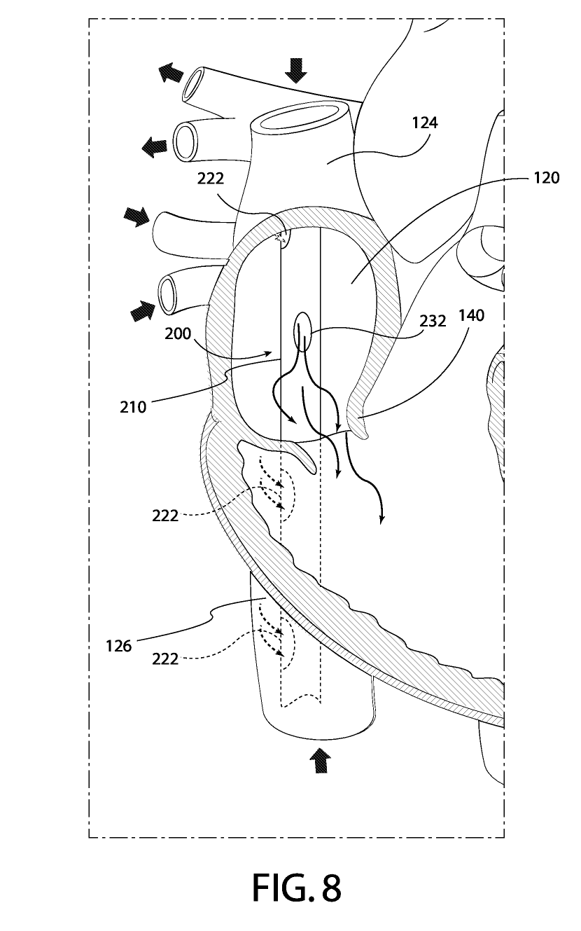

The distal end ultrasound transducer 260 may be supported by the tubular body 210 and may be disposed proximate the distal end 250 of the vascular device. As shown in FIG. 8, the vascular device 200 may be configured to be positioned such that the distal end 250 of the tubular body is disposed proximate a superior vena cava 124 of the patient's heart and a proximal portion of the vascular device is disposed proximate an inferior vena cava 126 of the patient's heart. Thus, in some embodiments, the distal end ultrasound transducer 260 may be configured to transmit and receive ultrasound signals so as to provide a view of an interior of the patient's blood vessel within which the vascular device 200 is disposed proximate the distal end, such that a longitudinal position of the vascular device may be determined with respect to the patient's blood vessel and surrounding anatomical structures to facilitate advancement of the vascular device to the correct position. For example, imaging provided by the distal end ultrasound transducer 260 may allow for identification of relevant vascular landmarks, including the cavo-atrial junction, innominate vein confluence, and/or tricuspid vein, among others, and may thus facilitate safe and precise cannula insertion and positioning, as described herein.

Accordingly, in some embodiments, images obtained from the distal end ultrasound transducer 260 and the second lumen port ultrasound transducer 240 may be used to guide the vascular device 200 to the vicinity of the right atrium 120 and to achieve the correct alignment of the second lumen port 232 with the tricuspid valve 140 such that optimal oxygenation efficiency may be obtained. For example, the image obtained using the distal end ultrasound transducer 260 may be referenced by the operator of the vascular device 200 to guide the vascular device from an entry point into the patient's vasculature (e.g., an entry point in the patient's groin area) up through the blood vessel to the inferior vena cava 126, through the inferior vena cava and into the right atrium 120, and past the right atrium into the superior vena cava 124. At this point, the operator of the vascular device 200 may reference the image obtained using the second lumen port ultrasound transducer 240 to fine-tune the longitudinal position of the vascular device by either moving the vascular device proximally or further advancing the vascular device distally, as well as to adjust the rotational position of the vascular device by rotating the device to align the first lumen port 232 with the tricuspid valve 140. In this regard, once in the operator sees an image from the second lumen port ultrasound transducer 240 that indicates the second lumen port 232 is in the right atrium, the operator may rotate the vascular device 200 until the tricuspid valve 140 is seen in the ultrasound image. The tricuspid valve 140 may be easily identified due to the opening and closing of the valve.

The images from the distal end ultrasound transducer 260 and the second lumen port ultrasound transducer 240 may be viewed by the operator at the same time, such as in side-by-side fashion, or sequentially, such as when the operator references the image from the distal end ultrasound transducer 260 for the first part of the insertion of the vascular device, then references the image from the second lumen port ultrasound transducer 240 for the adjustment and fine-tuning of the position of the vascular device.

Accordingly, in some embodiments, a method for positioning a vascular device, such as a vascular device according to the embodiments described above, within a patient's vasculature for use in ECMO is provided. A vascular device may initially be advanced from an entry point, through the patient's vasculature, and towards the patient's heart. As described above, the vascular device may comprise a tubular body defining a first lumen, a first lumen port in fluid communication with the first lumen, a second lumen, and a second lumen port in fluid communication with the second lumen. The first lumen may be configured to extract deoxygenated blood from the patient's vasculature via the first lumen port and convey the deoxygenated blood to an ECMO machine for oxygenation, whereas the second lumen may be configured to convey oxygenated blood from the ECMO machine and infuse the oxygenated blood into the patient's vasculature via the second lumen port. The vascular device may further comprise an ultrasound transducer supported by the tubular body and disposed proximate the second lumen port.

The vascular device may be positioned proximate the patient's right atrium, such as by advancing the vascular device distally from an entry point in the vasculature as described above. An image may be received from the ultrasound transducer supported by the vascular device of an interior of the patient's blood vessel within which the vascular device is disposed proximate the second lumen port of the vascular device. The vascular device may be rotated based on the image received, such that the second lumen port is substantially aligned with the tricuspid valve of the patient's heart to achieve optimal perfusion of the patient's body.

As described above, in some embodiments, the ultrasound transducer may be a second lumen port ultrasound transducer, and the vascular device may further comprise a distal end ultrasound transducer supported by the tubular body and disposed proximate the distal end of the vascular device. A second image may be received from the distal end ultrasound transducer of an interior of the patient's blood vessel within which the vascular device is disposed proximate the distal end of the vascular device. A longitudinal position of the vascular device may be adjusted based on the images provided by the distal end ultrasound transducer and/or the second lumen port ultrasound transducer to achieve optimal perfusion of the patient's body, as described above.

Embodiments of a vascular device and method are described above that provide built-in imaging for cannula insertion, such as cannula insertion for ECMO procedures. Embodiments of the device and method may greatly reduce, if not eliminate, the possibility of inadvertent arterial insertion, such an in cases in which the ultrasound transducer described above includes a Doppler module that indicates flow direction and velocity. Embodiments of the device and method may also significantly reduce the occurrence of cannula malpositioning, which most commonly involves positioning of the cannula across the tricuspid valve or into the hepatic veins, both of which are often fatal events. Additionally, embodiments of the vascular device and method may allow for more precise positioning of the first and second lumen ports, such as in embodiments of the vascular device in which the first and second lumens are defined by a single tubular body (e.g., in multi-port cannulas, which may rely more heavily on accurate port alignment). Embodiments of the invention as described above may thus facilitate the widespread adaptation of VV-ECMO in environments that lack the support necessary for traditional insertion techniques. For example, embodiments of the device and method may allow for cannulation to be performed by intensivists and may eliminate the need for surgeons, and cardiologists and/or other specialists to be present during the cannulation and preparation of a patient for ECMO. Furthermore, embodiments of the vascular device may improve patient safety and increase the efficacy of ECMO support by ensuring precise cannula positioning and may, in some cases, reduce the need for AV-ECMO, which can be a more risky procedure than VV-ECMO.

The devices and methods depicted in the figures and described above represent only certain configurations of the vascular device and method. The particular configurations and methods of delivery will depend on the patient's anatomy, the condition and location of the target site, the preferences of the practitioner, and other considerations. Although embodiments of a vascular device having a tubular body with multiple lumens (e.g., a single cannula that accomplishes both extraction of deoxygenated blood and infusion of oxygenated blood via different lumens), it is understood that embodiments of the device may be employed on one or more single lumen devices used in ECMO procedures.

For example, in other embodiments, a vascular device may be provided that is configured for placement within a patient's vasculature for use in ECMO, where the vascular device comprises a tubular body defining a lumen (e.g., a single lumen) and a port in fluid communication with the lumen. The lumen may be configured to extract deoxygenated blood from the patient's vasculature or infuse oxygenated blood into the patient's vasculature and may be in fluid communication with an ECMO machine. An ultrasound transducer may be supported by the tubular body and disposed proximate the port. In this regard, the ultrasound transducer may be configured to transmit and receive ultrasound signals so as to provide an image of an interior of the patient's blood vessel within which the vascular device is disposed proximate the location of the port, such that a position of the vascular device may be determined with respect to the patient's blood vessel and surrounding anatomical structures and such that the position of the vascular device may be adjusted by an operator of the vascular device based on the image provided by the ultrasound transducer to achieve optimal perfusion of the patient's body.

In still other embodiments, a vascular device may be provided that is configured for placement within a patient's vasculature for use in extracorporeal membrane oxygenation (ECMO), where the vascular device comprises a tubular body having a distal end and defines a lumen (e.g., a single lumen) and a port in fluid communication with the lumen. The lumen may be configured to extract deoxygenated blood from the patient's vasculature or infuse oxygenated blood into the patient's vasculature and may be in fluid communication with an ECMO machine. The vascular device may further comprise an ultrasound transducer supported by the tubular body and disposed proximate the distal end. The ultrasound transducer may be configured to transmit and receive ultrasound signals so as to provide an image of an interior of the patient's blood vessel within which the vascular device is disposed proximate the distal end, such that a position of the vascular device may be determined with respect to the patient's blood vessel and surrounding anatomical structures and such that the position of the vascular device may be adjusted by an operator of the vascular device based on the image provided by the ultrasound transducer to achieve optimal perfusion of the patient's body.

Many modifications and other embodiments of the invention will come to mind to one skilled in the art to which this invention pertains having the benefit of the teachings presented in the foregoing descriptions and the associated drawings. In this regard, one or more features described above and illustrated in the figures with respect to a particular embodiment may replace or be used in combination with other features described and illustrated with respect to other embodiments. For example, although the embodiment of FIGS. 4 and 5 is shown as including a wired transducer, this embodiment may be modified to incorporate the wireless transducer(s) shown in FIGS. 6 and 7, and vice versa. Moreover, although example ECMO procedures are described above to facilitate understanding of the invention, it is to be understood that embodiments of the vascular device described above may be used to implement various ECMO circuits and may make use of different insertion techniques, entry points, delivery devices, etc. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A vascular device configured for placement within a patient's vasculature, the vascular device comprising:
a tubular body defining:
a first lumen,
a first lumen port in fluid communication with the first lumen, wherein the first lumen is configured to extract deoxygenated blood from the patient's vasculature via the first lumen port and convey the deoxygenated blood to a machine for oxygenation,
a second lumen, and
a second lumen port in fluid communication with the second lumen, wherein the second lumen is configured to convey oxygenated blood from the machine and infuse the oxygenated blood into the patient's vasculature via the second lumen port; and
an ultrasound transducer supported by the tubular body and disposed proximate the second lumen port.

2. The vascular device of claim 1, wherein the tubular body defines a plurality of first lumen ports.

3. The vascular device of claim 2, wherein the tubular body defines 2 to 4 first lumen ports on a distal side of the second lumen port or 2 to 6 first lumen ports on a proximal side of the second lumen port.

4. (canceled)

5. The vascular device of claim 1, wherein the vascular device is configured to be positioned proximate a right atrium of the patient's body such that oxygenated blood flowing from the second lumen port is directed at the tricuspid valve of the heart.

6. The vascular device of claim 1, wherein the tubular body defines a distal end having an opening in fluid communication with the first lumen, such that deoxygenated blood is received into the first lumen from the patient's vasculature via the opening of the distal end and is conveyed to the machine for oxygenation.

7. The vascular device of claim 1, wherein the ultrasound transducer is a second lumen port ultrasound transducer, the vascular device further comprising a distal end ultrasound transducer supported by the tubular body and disposed proximate a distal end of the tubular body, wherein the distal end ultrasound transducer is configured to transmit and receive ultrasound signals so as to provide an image of an interior of the patient's blood vessel within which the vascular device is disposed proximate the location of the distal end, such that a longitudinal position of the vascular device may be determined with respect to the patient's blood vessel and surrounding anatomical structures and such that the longitudinal position of the vascular device may be adjusted by an operator of the vascular device based on the image provided by at least one of the distal end ultrasound transducer or the second lumen port ultrasound transducer to achieve optimal perfusion of the patient's body.

8. The vascular device of claim 1, wherein the vascular device is configured to be positioned such that a distal end of the tubular body is disposed proximate a superior vena cava of the patient's heart and a proximal portion of the vascular device is disposed proximate an inferior vena cava of the patient's heart.

9. The vascular device of claim 1, wherein the ultrasound transducer is wireless.

10. The vascular device of claim 1, wherein the ultrasound transducer is integrally formed with the tubular body.

11. A method for positioning a vascular device within a patient's vasculature, the method comprising:
advancing a vascular device from an entry point through the patient's vasculature towards the patient's heart, wherein the vascular device comprises:
a tubular body defining a first lumen, a first lumen port in fluid communication with the first lumen, wherein the first lumen is configured to extract deoxygenated blood from the patient's vasculature via the first lumen port and convey the deoxygenated blood to a machine for oxygenation, a second lumen, and a second lumen port in fluid communication with the second lumen, wherein the second lumen is configured to convey oxygenated blood from the machine and introduce the oxygenated blood into the patient's vasculature via the second lumen port, and
an ultrasound transducer supported by the tubular body and disposed proximate the second lumen port;
positioning the vascular device proximate the patient's right atrium;
receiving an image of an interior of the patient's blood vessel within which the vascular device is disposed proximate the location of the second lumen port of the vascular device from the ultrasound transducer supported by the vascular device; and
rotating the vascular device based on the image received, such that the second lumen port is substantially aligned with the tricuspid valve of the patient's heart to achieve optimal perfusion of the patient's body.

12. The method of claim 11, wherein the tubular body defines a plurality of first lumen ports.

13. The method of claim 12, wherein the tubular body defines 2 to 4 first lumen ports on a distal side of the second lumen port.

14. The method of claim 12, wherein the tubular body defines 2 to 6 first lumen ports on a proximal side of the second lumen port.

15. The method of claim 11, wherein the tubular body defines a distal end having an opening in fluid communication with the first lumen, such that deoxygenated blood is received into the first lumen from the patient's vasculature via the opening of the distal end and is conveyed to the machine for oxygenation.

16. The method of claim 11, wherein the ultrasound transducer is a second lumen port ultrasound transducer, the vascular device further comprising a distal end ultrasound transducer supported by the tubular body and disposed proximate a distal end of the vascular device, the method further comprising receiving a second image of an interior of the patient's blood vessel within which the vascular device is disposed proximate the distal end of the vascular device from the distal end ultrasound transducer.

17. The method of claim 16 further comprising adjusting a longitudinal position of the vascular device based on the images provided by the distal end ultrasound transducer and the second lumen port ultrasound transducer to achieve optimal perfusion of the patient's body.

18. The method of claim 11 further comprising positioning the vascular device such that a distal end of the vascular device is disposed proximate a superior vena cava of the patient's heart and a proximal portion of the vascular device is disposed proximate an inferior vena cava of the patient's heart.

19. The method of claim 11, wherein the ultrasound transducer is wireless.

20. The method of claim 11, wherein the ultrasound transducer is integrally formed with the tubular body.

21. A vascular device configured for placement within a patient's vasculature, the vascular device comprising:
a tubular body having a distal end and defining a lumen and a port in fluid communication with the lumen, wherein the lumen is configured to extract deoxygenated blood from the patient's vasculature or infuse oxygenated blood into the patient's vasculature and is in fluid communication with a machine for oxygenation; and
an ultrasound transducer supported by the tubular body and disposed proximate the port or the distal end.

22. (canceled)
